(19) United States
(12) Patent Application Publication
(10) Pub. No.: US 2005/0038381 A1
(43) Pub. Date: Feb. 17, 2005

(54) Catheter having a balloon member recessedly attached thereto

(75) Inventor: Donald J. McMichael, South Jordan, UT (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/639,057

(22) Filed: Aug. 11, 2003

(51) Int. Cl. A61M 29/00

(52) U.S. Cl. 604/96.01

(57) ABSTRACT

The present invention relates to catheters wherein at least one end of the balloon thereof is attached to the catheter shaft in a recess therein. Other aspects of the catheters will have both ends of the balloon recessedly secured. The recessed attachment of the balloon provides the opportunity to use a smaller stoma opening than has been necessary with prior catheters not having a recess.

### BACKGROUND

Catheterization of a body cavity is frequently performed in medical procedures either to insert substances into or to remove substances from the body. During many of these procedures, it is necessary to keep the catheter in a relatively stable position to perform the desired insertion or removal. With the use of enteral feeding catheters (i.e., catheters which enable the administration of nutritional solutions directly into the stomach or intestines), for example, it is necessary to ensure that the catheter is not accidentally removed from the stomach or intestines. This is true both during the actual administration or removal of fluids, and the time periods in between.

In order to ensure that a catheter is maintained in the proper position, it is common to use a balloon disposed near the distal (patient) end of the catheter shaft. Inflating the balloon causes the balloon to contact the anatomical structure (i.e., a duct or stomach wall) and thereby prevents the catheter from moving out of the proper position. In the case of enteral feeding, a stoma is formed leading into the stomach or intestine. The catheter is positioned to extend through the stoma so as to form a channel into the stomach or intestines through which enteral feeding solutions may be instilled.

FIG. 1 shows a side view of a prior art balloon catheter 10 having a head 14 disposed at a proximal end 15 of the catheter 10. The head 14 contains valves (not shown) which regulate the flow of fluids through the balloon catheter 10. The head 14 also prevents the balloon catheter 10 from completely advancing through the stoma and into the stomach or intestine of the user.

To prevent the catheter 10 from being pulled out of the stomach/intestinal wall, a balloon 18 is disposed along a catheter shaft 26. The catheter 10 is shown having an optional stiff tip 30, which is attached to the catheter shaft 26 at a distal end 17 thereof opposite the head 14. The catheter shaft 26 is typically made of a medical grade silicone. The stiff tip 30, when present, is also frequently formed of a medical grade silicone but is usually configured to be as rigid as or less rigid than the catheter shaft 26.

The balloon 18 is advantageous because it allows the catheter shaft 26 to be inserted into the stoma (not shown) while the balloon 18 is uninflated. Once the catheter shaft 26 is properly positioned in the stoma, a syringe (not shown) is inserted into a side port 36 of the head 14 and a fluid is injected into the balloon 18 through a lumen (not shown in FIG. 1) of the catheter 10 so as to inflate the balloon 18.

While the balloon 18 remains inflated, the catheter 10 stays properly positioned in the stoma. If the catheter 10 needs to be removed, the balloon 18 may be deflated so that it will not interfere with withdrawal of the catheter shaft 26 and stiff tip 30. The position of the balloon catheter 10 is maintained in such a manner until removal is desired. The type of balloon 18 shown in FIG. 1 is fashioned around the perimeter of the catheter shaft 26 such that when it is deflated it reduces or contracts about the shaft 26 but is still clearly larger than overall diameter of the catheter.

Attachment of the balloon 18 to the catheter shaft 26 is frequently accomplished by gluing the balloon proximal end 20 and the balloon distal end 22 to corresponding positions on the external surface of the catheter shaft 26 so as to form a proximal cuff 32 and a distal cuff 34, respectively. Such cuffs 32 and 34 are longitudinal sections of the balloon 18 whose inside diameters correspond to the outside diameter of the shaft 26 at their respective points of attachment to the catheter 10 and have a distance between them which is about the length of the uninflated balloon 18. The cuffs 32 and 34 must be of sufficient length to provide a tight and durable seal between the balloon 18 and the catheter shaft 26.

While the prior art balloon configuration shown in FIG. 1 works to maintain the balloon catheter 10 in the proper position within the patient, balloon catheters of this type as well as the other known balloon catheters do have disadvantages. For example, one drawback of prior balloon catheters is the attachment of the balloon 18 to the catheter 10 and the corresponding sizing issues. With regard to the catheter 10 of FIG. 1, the balloon 18 is attached to the catheter shaft 26, via proximal and distal cuffs 32 and 34, respectively, which extend out or away from the rest of the balloon 18 and which effectively increase the diameter of the shaft 26 at those points.

In use it is generally desirable to create as small a stoma as possible which will accommodate the catheter of choice yet will provide for a seal of the balloon 18 against the patient (not shown) so as to avoid leakage. The existence of proximal extending cuff 32 in prior catheters added to the diameter of the shaft 26 such that a larger stoma opening (not shown) is required. That is in prior catheters in order to obtain the necessary contact or seal between the balloon 18 and the patient (not shown), the proximal cuff 32 needed to be positioned on the external surface of the catheter shaft 26 such that at least a portion of the proximal cuff 32 is within the stoma (not shown). As the entire cuff extends or is located above the outer surface of the catheter shaft 26, a larger stoma opening is required than if the proximal cuff 32 did not extend or only a portion thereof extended or was located above the surface of the catheter shaft 26, as the effective diameter (diameter of shaft plus thickness of cuff at point of measurement) of the shaft is increased by a non-recessed attachment.

Another disadvantage of prior balloon catheters is discomfort to the user. In an attempt to reduce irritation of anatomical structures which come into contact with distal end of the catheter, some prior catheters wrap the distal cuff 34 of the balloon 18 around the tip 30 of the catheter shaft 26 and then attach the distal cuff 34 to the interior of the catheter shaft. However, in prior devices when the distal cuff 34 is secured to the interior of the catheter shaft 26 the thickness of the cuff reduced the effective inside diameter of the shaft. The reduction in effective inside diameter of the catheter shaft 26 results in lower flow rates therethrough. Accordingly, either longer feeding times are required or a larger catheter shaft must be substituted so that when the internal attachment of distal cuff 34 is effected the desired flow rate may still be achieved. Thus, either alternative has disadvantages.

Accordingly, there is a need in the art for a balloon catheter with the capability of interior balloon attachment to the catheter shaft without experiencing the difficulties and/or disadvantages associated with the prior devices.

An additional disadvantage of prior catheters such as those in FIG. 1 is that the edges 35 and 37 of the cuffs 32 and 34, respectively present another edge which can catch on tissue as it passes into a patient or which can otherwise cause or lead to irritation.

Accordingly, there is a need for a catheter which allows for the creation of a sufficient seal between the catheter and the patient without the need for an increase in stoma size. There is also a need in the art for a catheter which will reduce the amount of patient contact with or exposure to rough or sharp edges which may be associated with the attachment of a balloon or sleeve to the catheter, thereby reducing or eliminating patient irritation caused thereby.

### SUMMARY OF THE INVENTION

In response to the difficulties and problems discussed above, a catheter having an expandable member attached to the catheter shaft in a recess therein has been developed. More specifically, one aspect of this invention is directed to a catheter having an expandable member having a first end and a second end; and an elongate shaft having a first end, a second end, an exterior surface having a recess, a first lumen adapted for fluid communication and a second lumen adapted for fluid communication with the expandable member; wherein at least a portion of the first end of the expandable member is attached to the shaft in the recess. The shaft may have a second recess in which the second end of the expandable member is attached.

Another embodiment of the present invention is directed to a catheter having an expandable member and an elongate shaft. The expandable member has a first end and a second end, each of the ends of the expandable member having a thickness. The elongate shaft has a wall, a first lumen adapted for fluid communication and a second lumen adapted for fluid communication with the expandable member. The wall of the shaft has an outer surface and a recess. The wall further having a first thickness immediately proximal the recess and a second thickness in the recess. In this embodiment at least a portion of the first end of the expandable member is attached to the shaft in the recess such that the thickness of at least a portion of the first end of the expandable member and the second thickness of the wall in the recess is no greater than the first thickness of the wall.

The present invention is also directed to a catheter including an elongate shaft and an expandable member. The elongate shaft has a first and a second end. The expandable member has a proximal end and a distal end with respect to the first end of the of the shaft, each of the proximal and distal ends having a thickness. The elongate shaft further has a wall, the wall having an outer surface and a recess. The shaft has at least a first diameter immediately proximal the recess, a second diameter in the recess. The elongate shaft also includes a first lumen adapted for fluid communication and a second lumen adapted for fluid communication with the expandable member. In this aspect of the invention at least a portion of the proximal end of the expandable member is attached to the shaft in the recess such that the thickness of at least a portion of the proximal end of the expandable member and the second diameter of the shaft in the recess is less than about 1.15 times the first diameter of the shaft.

The present invention is also directed to a catheter including an expandable member having a first end and a second end, each of the ends of the expandable member having a thickness; and an elongate shaft. The elongate shaft includes a first lumen adapted for fluid communication, a second lumen adapted for fluid communication with the expandable member, and a wall having an outer surface, a recess. The wall also has at least a first diameter immediately proximal the recess and a second diameter in the recess. The catheter is such that when at least a portion of the first end of the expandable member is attached to the shaft in the recess such that at least a portion of the thickness of the first end of the expandable member and the second diameter of the wall in the recess is less than about 1.25 times the first diameter of the wall.

Another embodiment of the present invention is directed to a balloon catheter having a head with at least two openings through which fluid may be passed; a catheter shaft extending from the head; and a sleeve having a first end and a second end. The catheter shaft has a first and second lumen disposed in communication with the at least two openings, the catheter shaft further having an interior, an exterior, an outer diameter and a recess. Each end of the sleeve has a thickness. In this embodiment, at least a portion of the first end of the sleeve is attached in a recess along the shaft such that the outer diameter of the shaft at the recess plus the thickness of at least a portion of the first end of the sleeve attached in the recess is less than about 1.15 times the outer diameter of the shaft immediately proximal the recess. An expandable cavity may formed between the sleeve and the shaft.

Yet another aspect of the present invention is directed to a catheter configured for placement through a stoma into a body cavity such that the catheter may be maintained in the stoma. The catheter may generally include a head, an expandable sleeve having a first end and a second end each end having an edge, and an elongate shaft having at least a first lumen extending longitudinally therethrough, a first end, a second end, an interior, an inside diameter and a recess. At least a portion of one end of the sleeve is attached to the recess on the interior of the shaft such that the effective inside diameter of the shaft at the edge of the end of the sleeve is at least about 90% of the inside diameter of the shaft immediately proximal the recess.

Still another aspect of the present invention is directed to a catheter generally including a head, an expandable sleeve having a first end and a second end each end having an edge, and an elongate shaft having at least a first lumen extending longitudinally therethrough, a first end, a second end, an interior, an inside diameter and a recess. At least a portion of one end of the sleeve is attached to the recess on the interior of the shaft such that the effective inside diameter of the shaft at the edge of the end of the sleeve is no less than about 0.95, and desirably no less than about 0.97 times the inside diameter of the shaft immediately proximal the recess.

The invention will be more fully understood and further features and advantages will become apparent when reference is made to the following detailed description of exemplary embodiments of the invention and the accompanying drawings.

### BRIEF DESCRIPTION OF THE DRAWINGS

The above and other objects, features and advantages of the invention will become apparent from a consideration of the subsequent detailed description presented in connection with the accompanying drawings in which:

FIG. 1 shows a side view of a prior art balloon catheter, the balloon being in an inflated configuration.

FIG. 2 is a side view of one embodiment of the present invention, the catheter having the first end of the balloon or elongate sleeve attached to the catheter in a recess along the catheter shaft. The catheter is shown with an uninflated balloon.

FIG. 3 is a cross-sectional view of the catheter of FIG. 2.

FIG. 3A is an enlarged view of the portion of the catheter of FIG. 3 within the hashed circle;

FIG. 4 is a cross-sectional view of an expandable member having annular rings therein;

FIG. 5 is a cross-sectional view of a catheter of the present invention having both ends of an expandable member attached to the catheter shaft in recesses along catheter shaft;

FIG. 5A is an enlarged view of the portion of the catheter of FIG. 5 within the hashed circle;

FIG. 5B is an enlarged view of the portion of the catheter of FIG. 5 within the hashed circle, except that the expandable member has been removed to allow for a better view of one embodiment of a recess in the catheter shaft;

FIG. 6 is a cross-section of another embodiment of a catheter of the present invention having a tip at the distal end of the catheter to which an expandable member is attached;

FIG. 6A is an enlarged view of the portion of the catheter of FIG. 6 within the hashed circle;

FIG. 7 is a cross-sectional view of a catheter of the present invention having the distal end of an expandable member attached at the distal end of the catheter shaft;

FIG. 7A is an enlarged view of the portion of the catheter of FIG. 6 within the hashed circle; and

FIG. 8 is a cross-sectional view of a catheter of the present invention having a unitary component attached to the distal end of the catheter.

### DETAILED DESCRIPTION OF THE PRESENT INVENTION

Reference will now be made to the drawings in which the various elements of the present invention will be given numeral designations and in which the invention will be discussed so as to enable one skilled in the art to make and use the invention. It should be appreciated that each example is provided by way of explaining the invention, and not as a limitation of the invention. For example, features illustrated or described with respect to one embodiment may be used with another embodiment to yield still a further embodiment. These and other modifications and variations are within the scope and spirit of the invention.

It will be appreciated that while reference is made to an expandable member, the term expandable member is intended to mean or include, but is not limited to, a balloon, a sleeve, an inflatable member, an elastomeric sleeve, an expandable region or portion, an inflatable member, any other suitable expandable means, and the like. However, for ease of reading and understanding of this disclosure and not intending to be limited thereby, expandable member will hereinafter be referred to as a balloon. It will also be appreciated that throughout the disclosure reference is made to inflation of a balloon, however, the present invention is not intended to be limited only to inflation. That is, while inflation is used herein for purposes of ease of reading and understanding the disclosure, the term inflation is also intended to mean or include, but is not limited to, expansion, enlargement, swelling and the like.

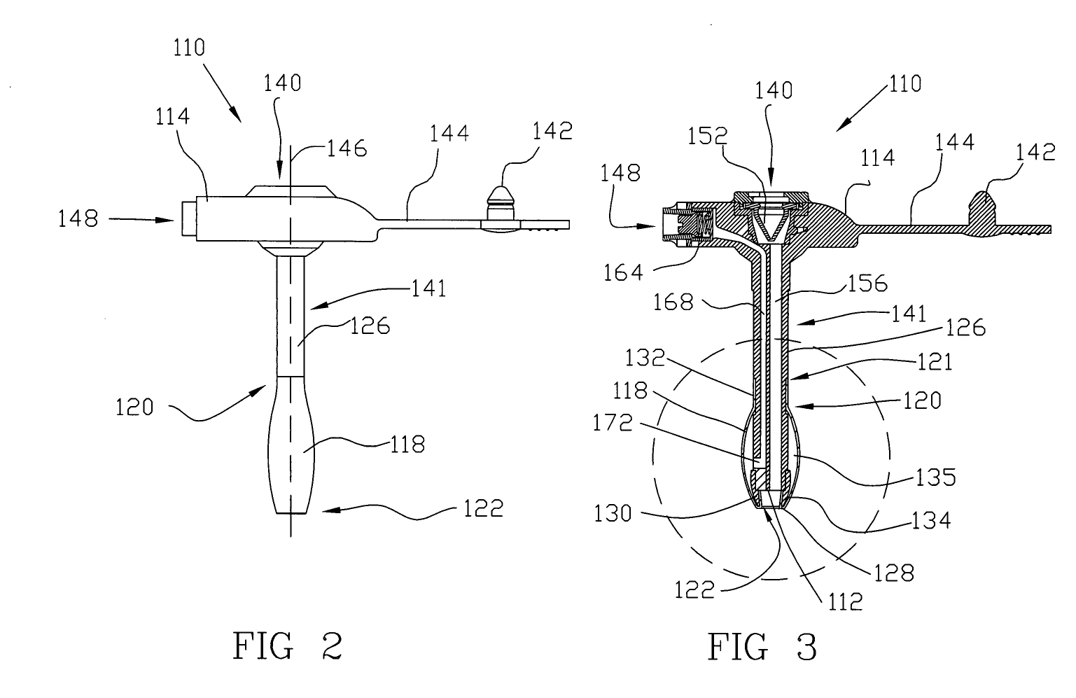

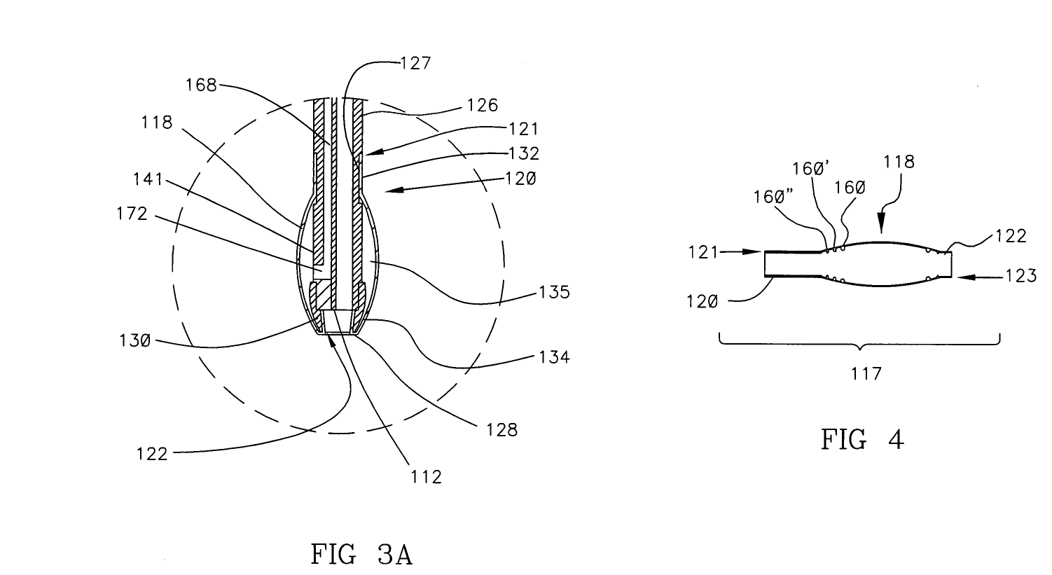

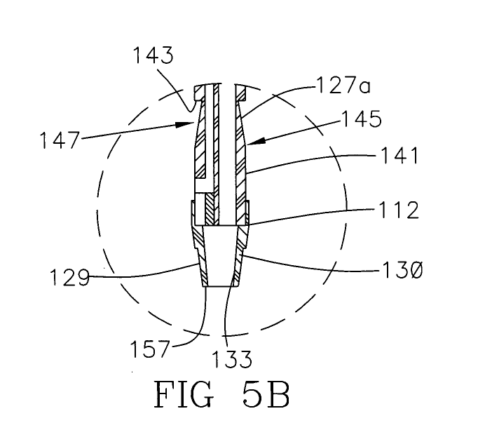

Referring now to FIGS. 2, 3, and 3A, there is shown a side view, a sectional view, and a partial sectional view, respectively, of a catheter 110 made in accordance with the teachings of the present invention. The catheter 110 generally includes a head 114 (FIGS. 2 and 3), a balloon 118, and a catheter shaft 126. The head 114 (FIGS. 2 and 3) has a opening 140 (FIGS. 2 and 3) to a feeding lumen 156 (FIGS. 3 and 3A) within the shaft 126, for bolus feeding or providing other nutrient fluids, formula, and the like to a patient (not depicted). The catheter 110 (FIGS. 2 and 3) also shows an optional stiff tip 130 (FIGS. 3 and 3A) attached at the distal end 112 (FIGS. 3 and 3A) of the catheter shaft 126. The stiff tip 130 (FIGS. 3 and 3A) has an interior surface 133 (FIGS. 5-7A) which defines a passageway which is configured for the passage of fluids, solutions, certain solids, or the like therethrough and into or out of the catheter 110 (FIGS. 2 and 3). An anti-reflux valve 152 (FIG. 3), which is generally included to prevent back-flow of the nutrient formula, is shown disposed between the opening 140 (FIGS. 2 and 3) and the inflation lumen 168 (FIGS. 3 and 3A). Inflation port 148 (FIGS. 2 and 3) is disposed in the head 114 (FIGS. 2 and 3) and communicates with inflation lumen 168 (FIGS. 3 and 3A) which extends longitudinally through the shaft 126. The inflation lumen 168 (FIGS. 3 and 3A) is shown terminating laterally to the shaft 126 at port 172 (FIGS. 3 and 3A) into the cavity 135 (FIGS. 3 and 3A) created by the balloon 118 and the shaft 126.

A one-way valve 164 (FIG. 3) may be disposed between the inflation port 148 and inflation lumen 168 (FIGS. 2 and 3A). Application of positive fluid pressure, such as with air or saline, within and/or upon the inflation lumen 168 (FIGS. 2 and 3A) by way of the inflation port 148 (FIGS. 2 and 3) may cause the balloon 118 to inflate as the fluid fills the cavity 135 (FIGS. 3 and 3A) between the balloon 118 and the catheter shaft 126. Valve 164 (FIG. 3) helps prevent inadvertent deflation of the balloon 118. Also shown associated with the head 114 (FIGS. 2 and 3) is a plug 142 (FIGS. 2 and 3) for the opening 140 (FIGS. 2 and 3) and a lanyard 144 (FIGS. 2 and 3) for retaining the plug 142 in a ready position. The plug 142 can be inserted in the opening 140 thereby reducing or precluding contamination when the opening 140 is not in use. Feeding lumen 156 (FIGS. 3 and 3A) extends longitudinally through shaft 126 and is shown terminating at the second or distal end 112 (FIGS. 3 and 3A) of the shaft 126. It will be appreciated that the terms “proximal” and “distal” are used herein solely for purposes of discussion and ease of understanding, but are generally used herein to describe a position of an element relative to the head of a desired embodiment of the present invention such as that illustrated in FIGS. 2, 3, 5, 6, 7 and 8.

It will be appreciated that the size and shape of the cavity 135 (FIGS. 3 and 3A) defined by or between the exterior 141 (FIGS. 2-3A and 5B) of the shaft 126 (FIGS. 2-3A, 5, 6 and 7) and the balloon 118 (FIGS. 2-7A) may be varied during production or controlled by the user or clinician during use. Additionally, as discussed in more detail below the balloon 118 of the catheter 110 (FIGS. 2 and 3) may be designed to have a certain size and/or shape in either or both of its inflated or uninflated configurations. It will be understood and appreciated that varying the length of the balloon 118 and/or the points along the shaft 126 at which the ends 120 (FIGS. 2-4) and 122 (FIGS. 2-4) of the balloon 118 (FIGS. 2-7A) are attached may affect the shape of the resulting balloon.

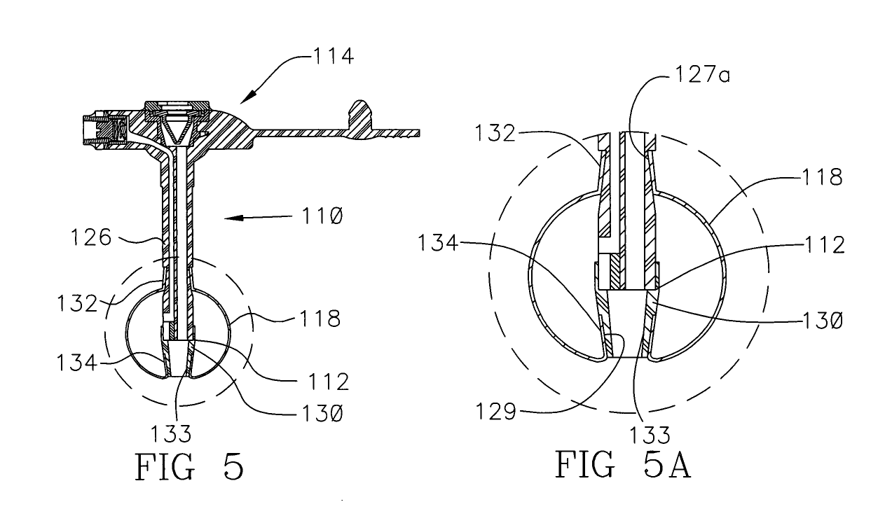

Another suitable way of controlling the shape of the resulting inflated balloon includes, but is not limited to, annular rings, such as those shown at 160, 160′, and 160″ in FIG. 4, which may limit or promote expansion of the balloon in one or more directions. Other suitable ways of controlling the shape of the resulting balloon include, but are not limited to, those discussed in the U.S. Pat. No. 6,264,631 B1 to Willis et al., which is incorporated by reference in its entirety. For example, a plurality of annular rings may be disposed about the proximal end of the balloon. These rings force a greater volume of inflation medium into the distal end of the balloon, thus urging it to distend in the direction of the tip. In another embodiment, a plurality of centrally located annular rings bias radially against inflation of the balloon. These forces cause the balloon to distend longitudinally. The proximal distention is limited by the proximal body cavity wall. Thus, the distention over the distal tip is even more pronounced. In yet another embodiment, the uninflated balloon is longer than the portion of the shaft to which it is attached. This creates a longitudinal excess that allows the balloon to overlap the balloon cuffs and distend longitudinally upon inflation. As in the previously described embodiment, the proximal wall of the balloon may enhance distal distention of the balloon. In still yet another exemplary embodiment, the balloon may have a thicker portion or wall at the proximal end and a thinner portion or wall at the distal end. In such an embodiment, the natural bias of the balloon adjacent the proximal cuff urges the bulk of the inflation to occur adjacent the distal cuff where the natural bias is relatively weak.

The various components of balloon catheter 110 (FIGS. 2, 3, 5, 6 and 7) may be made of any suitable material and may desirably be formed from bio-compatible materials such as medical grade silicone or the like. And while valves 152 (FIG. 3) and 164 (FIG. 3) may be formed of any suitable material they are desirably made of a suitable polymer such as polycarbonate.

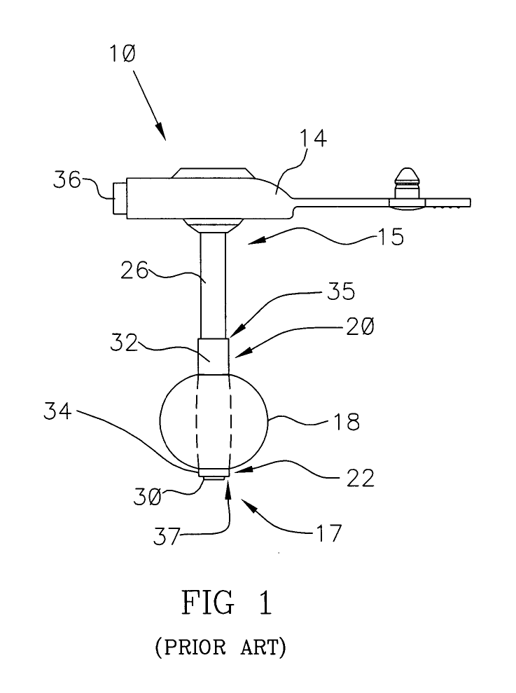

Unlike the prior art balloon catheter 10 shown in FIG. 1 which includes an attachment (such as first or proximal cuff 32) which extends in a generally longitudinal fashion above or adding to the diameter of the catheter shaft 26 away from the portion of the balloon 18 which expands, the first or proximal end 120 of the balloon 118 of the embodiment of the present invention shown in FIGS. 3 and 3A is attached to the catheter 110 in a recess 127 (FIG. 3A). That is, while the first end 120 of the balloon 118 may extend, at least in part, longitudinally along the catheter shaft 126 so as to be coaxial therewith, and so as to form, for example, a first cuff 132, the catheter shaft 126 desirably has a recess 127 (FIG. 3A) therein capable of accommodating or receiving at least a portion of the first cuff 132 of the balloon 118. While the recess 127 (FIG. 3A) may be of a variety of dimensions it is desirable that the recess 127 be capable of receiving at least the edge 121 (FIGS. 3-4) of the first cuff 132 (FIGS. 3 and 3A), and more desirably capable of receiving the entire first cuff 132. In either case it is desirable to minimize or avoid space about the first cuff 132 in the recess 127 (FIG. 3A). It is desirable for the edge 121 (FIGS. 3-4) of the cuff 132 (FIGS. 3 and 3A) to be positioned flush against the proximal edge 143 (FIG. 3) of the recess 127 (FIG. 3A) closest to head 114 (FIG. 3). The minimization or avoidance of space about the first cuff 132 (FIGS. 3 and 3A) and more specifically the edge 121 (FIGS. 3-4) of the first cuff 132 will reduce the number of edges which may contact the patient and which could cause irritation and/or provide a surface for bacteria to reside on as discussed in more detail below. It will be appreciated that while in some embodiments the recess will be generally flat or parallel relative to the central axis 146 (FIG. 2) of the catheter shaft 126 (FIGS. 2-3A, 5, 6 and 7), in other embodiments, the recess 127a (FIG. 5A) may be tapered so as to provide a gradual transition from the edge 143 (FIG. 5B) of the recess 127a (FIGS. 5A and 5B) closest to the head 114 (FIG. 5) to the opposite end 145 (FIG. 5B) of the recess 127a (FIGS. 5A and 5B). The taper may be such that bottom surface 147 (FIG. 5B) of the recess 127a merely blends back into or merges with the exterior surface 141 of catheter shaft 126 such that the end 145 (FIG. 5B) of the recess 127a opposite edge 143 (FIG. 5B) of the recess 127a may not be visible or readily discernible (as suggested in FIG. 5B).

The benefits of an end of a balloon which is fully received within a recess as well as the receipt of any portion of an end of a balloon will be appreciated; nevertheless some of those benefits are discussed below.

Having described at least one desired embodiment of the present invention it should be appreciated that a catheter of the present invention is desired to provide a catheter with a smaller effective outer diameter and/or a catheter which provides for the internal attachment of an end of a balloon yet does not reduce the potential flow rate through the catheter or necessitate a larger catheter to achieve the flow rate originally achievable where internal attachment was not present.

As suggested herein, it is desirable that the combination of the outer diameter of the recess in the catheter and the thickness of the end of the balloon be such that the total is not greater than, and more desirably less than, the outer diameter of the catheter shaft immediately proximal to the recess. Such desired dimensions may aid in the placement of the catheter and in some embodiments reduce the number or size of edges (such as edge 121 in FIGS. 3 and 3A) present which can cause irritation or provide a surface for bacteria growth as discussed in more detail herein.

Although in most embodiments it may be desirable for an end of the balloon to be fully recessed along the catheter shaft, the present invention also contemplates that in some embodiments that an end of the balloon may be only partially recessed relative to the surface of the catheter shaft. For example, in certain embodiments of the present invention, especially those being of smaller French sizes, such as pediatric devices, it may not be possible to fully recess an end of a balloon because of the limited inner and/or outer diameters of the catheter shaft, the thickness of the wall of the catheter shaft, and/or because the balloon end or cuff need be of a minimum size or thickness in order to effectively create the necessary seal. Additionally, it may not be necessary to fully recess the end or cuff of a balloon as in at least some embodiments an entire end or cuff of the balloon may not be positioned in the stoma when the catheter is properly positioned. That is, in one or more embodiments, the first end 120 (FIGS. 2-4) of the balloon 118 (FIGS. 2-7A) will have a certain length dimension and whether or not the entire end 120 is attached in a recess 127 (FIG. 3A) along the catheter shaft 126 (FIGS. 2-3A, 5, 6 and 7), when the catheter is positioned in a patient (not shown) only a portion of that length of the end 120 proximal the edge 121 (FIGS. 3 and 3A) may be in the stoma. Thus, while it is desirable for the effective outside or outer diameter of all portions (e.g. shaft, tip, etc.) of the catheter distal to edge 143 (FIGS. 3 and 5A) of the recess 127 (FIG. 3A) to be smaller than the diameter of the shaft 126 (FIGS. 2-3A, 5, 6 and 7) immediately proximal the recess 127 the effective outer diameter of the shaft 126 and end 120 (FIGS. 2-4) of the balloon 118 (FIGS. 2-7A) only needs to be less at at least one point along the recess than that which it would have been were no recess present. That said, it is also desirable for the thickness of the first end 120 (FIGS. 2-4) of the balloon 118 and the second diameter of the wall in the recess 127 (FIG. 3A) to be less than about 1.25, more desirably less than about 1.15 times the first diameter of the wall immediately proximal the recess. In certain embodiments or applications it may be desirable for the thickness of the first end 120 (FIGS. 2-4) of the balloon 118 and the second diameter of the wall in the recess 127 (FIG. 3A) to be less than about 1.1, less than about 1.05, less than about 1, less than about 0.95, or even more desirably less than about 0.9 times the first diameter of the wall immediately proximal the recess. It will be appreciated that these dimensions may be easiest to observe when the balloon is in an uninflated state. Further, it will appreciated that while the expandable portion of the balloon may exceed the above dimensions, if the ends 120, 122 or cuffs 132, 134 which are attached to the catheter shaft 126 fall within the above dimensions then that embodiment is deemed to be within the scope of the claims of the present invention. As noted herein, the above dimensions may only be present at at least one point along the length of the cuff in the recess (e.g. at the most proximal point of attachment of the balloon in the recess), but it is desirable for the dimensions to be present along a majority of the length of the cuff or end attached in the recess.

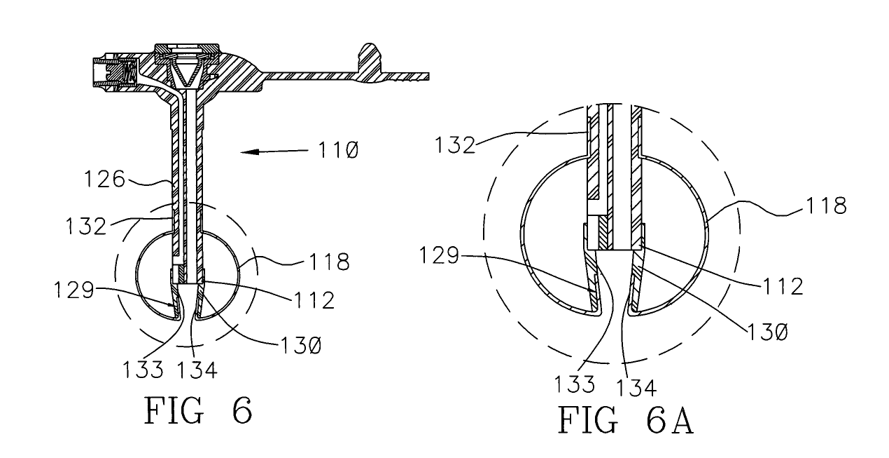

The second end 122 (FIGS. 2-4) of the balloon 118 (FIGS. 2-7A) may be secured to the catheter 110 (FIGS. 2, 3, 5, 6 and 7) in a variety of manners including, but not limited to, those similar to that used to attach the first end 120 (FIGS. 2-4) of the balloon 118 (FIGS. 2-7A). Thus, the attachment of the second end 122 (FIGS. 2-4) of the balloon 118 to the catheter shaft 126 (FIGS. 2-3A, 5, 6 and 7) may also be done in a recess 129 such as, for example, those shown in FIGS. 5A and 6A. Not all embodiments of the present invention require that the second end 122 (FIGS. 2-4) of the balloon 118 be attached in a recess 129 (FIGS. 5A and 6A) or that the second end 122 of the balloon 118 be attached to the catheter shaft 126. As such any suitable manner of directly or indirectly attaching the second end 122 of the balloon 118 to a catheter 110 (FIGS. 2 and 3) of the present invention is contemplated by the present invention.

In addition to being capable of attachment to a catheter 110 in a variety of manners, it will be appreciated that a balloon 118 may be attached to a catheter 110 (FIGS. 2, 3, 5, 6 and 7) in a variety of locations. It will also be appreciated that the size of the catheter 110 (FIGS. 2, 3, 5, 6 and 7) as well as the length (inflated and uninflated) of the balloon 118 (FIGS. 2-7A) may be varied in accordance with the size and shape of the body cavity (not shown) the catheter 110 is to be used in and the nature of the matter to be moved through the catheter 110. That is, in some instances, it may be desirable to use catheters having larger and/or wider shafts than in other embodiments.

Additionally, it will be appreciated that the provision of a recess 129 (FIGS. 5A and 6A) along the interior of the catheter shaft 126 as in FIGS. 5-6A does not necessarily mean that the inner diameter of the catheter shaft 126 need be any smaller than in those catheters not having a recess in accordance with the present invention, nor does the provision of a recess 129 (FIGS. 5A and 6A) along the interior surface 133 (FIGS. 5-7A) of the catheter shaft 126 necessitate the use of a catheter having a shaft with a larger outside diameter than non-recessed embodiments.

As suggested above, it will appreciated that in some embodiments it may be necessary to provide a recess which may not fully accommodate the thickness of a cuff (e.g. 132 or 134) of the balloon 118; however, it will also be appreciated that a recess that allows partial concealment of an end or cuff of the balloon is still an improvement over those prior catheters which provide no recess. Specifically, those embodiments of the present invention having a recess which provides a partial concealment of the balloon end along the outer surface 141 (FIGS. 2-3A) of the catheter shaft 126 exhibit advantages over current catheters as some reduction in effective outside diameter provides for a potential reduction in the minimum stoma size. It has been observed in at least one embodiment that the use of a recess along the outside surface 141 of the catheter shaft 126 has resulted in the ability to use a catheter size having dimensions which are about two French sizes (about 0.013 inches each) smaller than that which is possible with a currently available device not having a recess. And, those embodiments of the present invention having a recess 129 (FIGS. 5A and 6A) which provides for at least a partial concealment of the balloon end along the inner surface 133 (FIGS. 5-7A) of the catheter shaft 126 (FIGS. 2-3A, 5, 6 and 7) offer the opportunity for a greater flow rate through the shaft 126 (FIGS. 2-3A, 5, 6 and 7) than if no recess and no concealment of the balloon end 122 (FIGS. 2-4) were present. And, in those embodiments where a minimum inside diameter is required, the outside diameter may not have to be increased as much as current catheters having internal attachment of the balloon without the benefit of a recess. Again, where an end of the balloon is attached to a recess 129 (FIGS. 5A and 6A) on the interior or inner surface 133 (FIG. 5-6A) of the shaft 126 (FIGS. 2-3A, 5, 6 and 7) it is desirable that the effective inside diameter of the shaft 126 (and balloon at the most proximal point of attachment) be at least about 90% of the inside diameter of the shaft 126 immediately proximal the recess 129 (FIGS. 5A and 6A). More desirably, an end of the balloon may be attached to a recess on the interior surface 133 (FIGS. 5-7A) of the shaft such that the effective inside diameter of the shaft 126 (FIGS. 2-3A, 5, 6 and 7) at the most proximal point of balloon attachment (e.g. at the edge 123 of the end 122 of the balloon 118 as suggested in FIGS. 2-5A and 6-7A) is no smaller than about 0.95 times, and even more desirably no smaller than about 0.97 times the inside diameter of the shaft immediately proximal the recess 129. As with recesses on the outside of the shaft, the interior recesses may not be uniform in depth along the length of the catheter shaft but desirably provide at least some improvement over the prior catheters not having a recess.

As suggested above, the present invention also contemplates the inclusion of a tip 130 (FIGS. 3, 3A, 5-6A and 8) which may be attached to the second end 112 (FIGS. 3 and 3A) of the shaft 126. It is contemplated that the tip 130 may be part of the shaft (e.g., formed integrally with the shaft) or may be a separate attachment. Where a tip 130 is present the second or distal end 122 (FIGS. 2-4) of the balloon 118 (FIGS. 2-7A) may be attached to the tip 130 or to the shaft 126. When present either as an integral part of the catheter shaft 126 or separately formed and later attached, the tip 130 should be considered to be a part of the catheter 110. That is, for example, each embodiment should be considered to be attached to the catheter whether attached directly to the catheter shaft or the tip. The same is contemplated for interior attachment to the shaft 126 or tip 130 of a catheter.

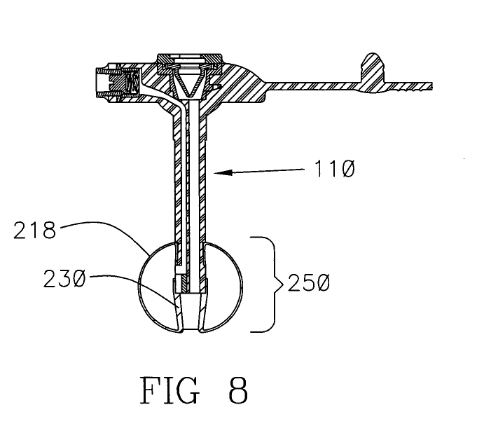

While there has been discussion above concerning attachment of a balloon to the interior 133 (FIGS. 5-7A) or to the exterior 141 (FIGS. 2-3A) of catheter shaft 126 (FIGS. 2-3A, 5, 6 and 7), the second end 122 (FIGS. 2-4) of the balloon 118 (FIGS. 2-7A) may alternatively be attached (not shown) to the distal end 157 (FIG. 5B) of the tip 130 (FIGS. 3, 3A, 5-6A and 8) of the catheter 110. Further still, rather than necessitating that the tip 130 and the balloon 118 be created separately from one another, it is also contemplated that the tip may be part of a unitary component 250 (FIG. 8), where the unitary component 250 includes a tip 230 integrally formed with a balloon 218. A more detailed description and discussion of exemplary unitary components may be found in commonly owned and copending U.S. patent application Ser. No. 10/306,999 (Attorney Docket No. 17,110A), Ser. No. 10/306,992 (Attorney Docket No. 17,110B), and Ser. No. 10/306,994 (Attorney Docket No. 17,110C), entitled “CATHETER WITH UNITARY COMPONENT”, “PROCESS FOR SECURING A TIP MEMBER TO CATHETER DURING PRODUCTION OF THE TIP MEMBER” and “PROCESS FOR PRODUCING UNITARY COMPONENT AND A CATHETER HAVING A UNITARY COMPONENT”, respectively, each to McMichael et al., and each filed Nov. 30, 2002, all of which are incorporated herein by reference in their entirety.

As will be appreciated, the catheters described above as well as those contemplated to be within the scope of the disclosure and claims of the present invention have several advantages over those prior catheters. For example, as a result of the attachment of the proximal end 120 (FIGS. 2-4) of the balloon 118 (FIGS. 2-7A) in a recess 127 (FIG. 3A) along the catheter shaft 126 (FIGS. 2-3A, 5, 6 and 7), it may be possible for users of catheters of the present invention to utilize smaller stoma openings (not shown) as the first end 120 (FIGS. 2-4) and/or cuff 132 (FIGS. 3 and 3A) of the balloon 118 will no longer add to or add as much to the effective outside diameter of the shaft 126 as with previous devices. Specifically, presuming for comparison purposes that the balloons and catheters of the present invention and a comparative prior device are otherwise the same with the exception of a recess in a catheter of the present invention, when the end of the balloon is attached in a recess along the catheter shaft the effective outside diameter (i.e. diameter of the shaft at the point of attachment of the balloon plus the thickness of the end of the balloon) of the present invention is desirably less than the outside diameter at the point attachment of the end of the balloon and the shaft of the prior device not having a recess. Thus, as the effective diameter of the catheter shaft may be smaller than with prior devices, one skilled in the art will appreciate that a smaller stoma opening may be used. Stated another way, as the creation of the necessary seal between the patient (not shown) and a catheter 110 (FIGS. 2-3A) not having all or part of cuff 132 (FIGS. 3 and 3A) of the balloon 118 (FIGS. 2-7A) above the exterior surface 141 (FIGS. 2-3A) of the catheter shaft 126 in the recess 127 (FIG. 3) in the stoma (not shown) when the catheter 110 is properly positioned the minimum stoma size may be reduced in some instances. As suggested above, a reduction in stoma size of up to about 2 French sizes (Note: 1 French size equals about 0.013 inches in diameter) has been achieved to date through the use of at least one embodiment of the present invention. Although not all decreases in stoma sizes will be as significant it will be appreciated that any reduction offers advantages over the current devices.

Additionally, by avoiding or minimizing the amount of a balloon cuff 132 (FIGS. 3 and 3A) in the stoma of the patient (when properly positioned) which extends above the exterior surface 141 (FIGS. 2-3A) of the catheter shaft 126 (FIGS. 2-3A, 5, 6 and 7) the balloon 118 of the present invention may be able to provide a better seal with the patient. At least one reason a better seal may be provided is that the stoma (not shown) may be smaller and thus fluids (if present at the stoma site) now have a smaller opening to pass through.

Further still, the use of a catheter having one or more ends 120, 122 attached to the catheter shaft 126 (FIGS. 2-3A, 5, 6 and 7) in a recess 127 (FIG. 3A), 129 (FIGS. 5A and 6A) thereon can result in less irritation upon the insertion and/or removal of the device as well as potentially reducing infection. That is, for example, at each end 120 (FIGS. 2-4) or 122 (FIGS. 2-4) of a balloon 118 (FIGS. 2-7A), there is an edge 121 (FIGS. 3-4) or 123 (FIG. 4), respectively. Thus, when at least one end 120 (FIGS. 2-4) or 122 (FIGS. 2-4) of a balloon 118 of the present invention is attached to the catheter shaft 126 (FIGS. 2-3A, 5, 6 and 7) in a recessed or partially recessed fashion, at least one edge 121 (FIGS. 3-4) or 123 (FIG. 4) of the balloon 118 will not be exposed or may have less exposure to the patient. This is of note as the prior art catheters typically had two fully exposed edges which could catch on tissue as they passed into or out of a patient or which could otherwise cause or lead to irritation. As a result of the ends 120 (FIGS. 2-4) or 122 (FIGS. 2-4) of the balloon 118 being recessed and/or inverted as shown and discussed herein, the catheters of the present invention will reduce, if not eliminate, a patient's contact with or exposure to potentially rough edges at the end 120 or 122 of a balloon 118. This reduction in contact or exposure may lead to a reduction or elimination of patient irritation caused by previous attachments or cuffs 32, 34 (FIG. 1) or their associated edges 35, 37 (FIG. 1). The reduction or elimination of exposed edges will also reduce the available surface area for bacteria and the like to accumulate and/or grow on and thus the potential for infection may also be reduced with the catheters of the present invention.

It will be appreciated that it is not intended that the use of a balloon which has one or more of its ends attached to or in a recess along a catheter shaft can and should only be used with catheters which are sized no larger than necessary to accommodate the balloon and/or its ends, rather the present invention provides the ability to use catheter shafts having smaller effective outer diameters than prior devices should that be of interest to the clinician.

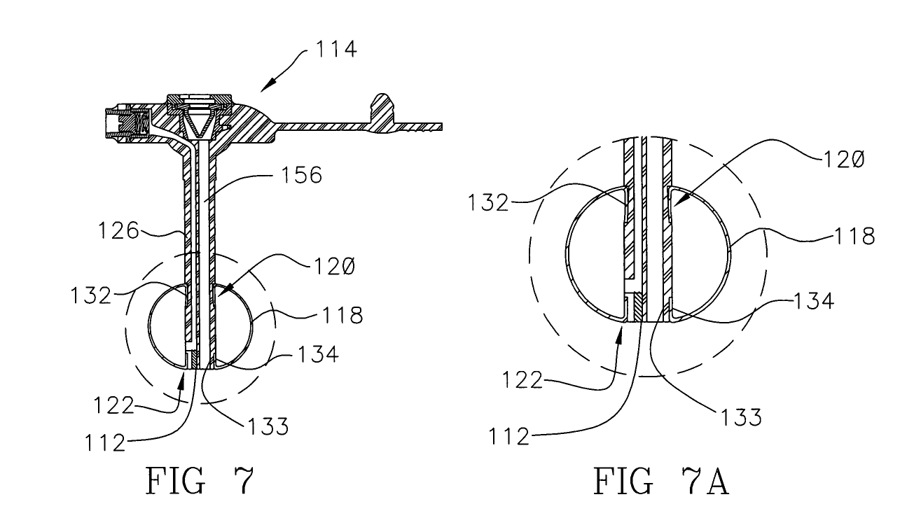

Further still, while the ends of a balloon may be attached in recesses on catheter shafts having a variety of lengths, a balloon having one or more ends invertedly attached (as shown in FIGS. 7-8) to a catheter shaft is suited for use with shorter catheter shafts than were previously possible with the prior art balloon catheters. While the shaft of a catheter of the present invention will need to be of a length sufficient to accommodate the balloon and enable the catheter's purpose the catheter need be no longer. That is, the inversion of one or more of the ends of the balloon 118 may reduce the length of shaft 126 which is necessary, as compared with prior devices, as the shaft of the present invention need not have additional length added to the shaft so as to accommodate balloon cuffs which extend away from the remainder of the balloon at both ends as is the case with prior devices. As suggested above, this reduction of catheter shaft length may decrease or minimize irritation associated with prior art devices. That is, as the catheter shaft 126 is shorter, the distal end 112 (FIGS. 3 and 3A) or tip 130 (FIGS. 3, 3A, 5-6A and 8) (depending on the embodiment) of the catheter 110 (FIGS. 2, 3, 5, 6 and 7) may be less likely to come in contact with the opposite side of the body cavity (not shown) into which the catheter is placed, and therefore less likely to cause irritation and/or discomfort associated with such contact. It will be appreciated that where an inverted end of the balloon is attached in a recess along the catheter, the recess may still reduce the minimum stoma size depending on the size and shape of the remainder of the catheter and the balloon. Further the recess may be sufficiently sized so as to accommodate the inverted attachment of the balloon as well as the expandable portion of the balloon and still reduce the effective outer diameter of the catheter or at least without the need to increase the stoma size needed to accommodate such an embodiment without the inverted attachment.

It will be appreciated that each embodiment of the present invention may not possess each and every component described or contemplated hereby and/or may not possess each and every advantage described or contemplated herein but all such embodiments are nevertheless contemplated to be within the scope of the disclosure and the attached claims.

While various patents and other reference materials have been incorporated herein by reference, to the extent there is any inconsistency between incorporated material and that of the written specification, the written specification shall control. In addition, while the invention has been described in detail with respect to specific embodiments thereof, those skilled in the art, upon obtaining an understanding of the invention, may readily conceive of alterations to, variations of, and equivalents to the described embodiments. It is intended that the present invention include such modifications and variations as come within the scope of the appended claims and their equivalents.

1. A catheter comprising:
an expandable member having a first end and a second end; and
an elongate shaft having a first end and a second end, an exterior surface having a recess, a first lumen adapted for fluid communication and a second lumen adapted for fluid communication with the expandable member;
wherein at least a portion of the first end of the expandable member is attached to the shaft in the recess.

2. The catheter of claim 1 wherein the shaft has a second recess in which at least a portion of the second end of the expandable member is attached.

3. The catheter of claim 1 the shaft further comprising an interior surface having a recess, wherein at least a portion of the second end of the expandable member is attached to the recess on the interior surface of the shaft.

4. The catheter of claim 1 wherein the second end of the shaft further comprises a tip.

5. The catheter of claim 4 wherein the tip has a recess in which at least a portion of the second end of the expandable member is attached.

6. The catheter of claim 4 wherein the tip may be part of the shaft or a separate attachment.

7. The catheter of claim 4 wherein at least a portion of the second end of the expandable member is attached to the tip.

8. The catheter of claim 1 wherein at least a portion of at least one of the ends of the expandable member forms a cuff.

9. The catheter of claim 1 further comprising a unitary component having a tip integrally formed with the expandable member, wherein the tip is attached to the second end of the shaft.

10. The catheter of claim 1 wherein the expandable member is a balloon or a sleeve.

11. A catheter comprising:
an expandable member having a first end and a second end, each of the ends of the expandable member having a thickness; and
an elongate shaft having:
a wall, the wall having an outer surface, and a recess, the wall having at least a first thickness immediately proximal the recess and a second thickness in the recess;
a first lumen adapted for fluid communication and a second lumen adapted for fluid communication with the expandable member;
wherein at least a portion of the first end of the expandable member is attached to the shaft in the recess such that the thickness of at least a portion of the first end of the expandable member and the second thickness of the wall in the recess is no greater than the first thickness of the wall.

12. The catheter of claim 11 wherein at least a portion of the first end of the expandable member is attached to the shaft in the recess such that the thickness of at least a portion of the first end of the expandable member and the second thickness of the wall in the recess is less than the first thickness of the wall.

13. A catheter comprising:
an expandable member having a first end and a second end, each of the ends of the expandable member having a thickness; and
an elongate shaft having:
a wall, the wall having an outer surface, and a recess, the shaft having at least a first diameter immediately proximal the recess and a second diameter in the recess;
a first lumen adapted for fluid communication and a second lumen adapted for fluid communication with the expandable member;
wherein at least a portion of the first end of the expandable member is attached to the shaft in the recess such that at least a portion of the thickness of the first end of the expandable member and the second diameter of the shaft in the recess is less than about 1.25 times the first diameter of the shaft.

14. A catheter comprising an elongate shaft having a first and a second end; and an expandable member having a proximal end and a distal end with respect to the first end of the of the shaft, each of the proximal and distal ends having a thickness, the elongate shaft further having a wall, the wall having an outer surface, and a recess, the shaft having at least a first diameter immediately proximal the recess, a second diameter in the recess, and a first lumen adapted for fluid communication and a second lumen adapted for fluid communication with the expandable member, wherein at least a portion of the proximal end of the expandable member is attached to the shaft in the recess such that at least a portion of the thickness of the proximal end of the expandable member and the second diameter of the shaft in the recess is less than about 1.15 times the first diameter of the shaft.

15. The catheter of claim 14 wherein at least a portion of the proximal end of the expandable member is attached to the shaft in the recess such that at least a portion of the thickness of the proximal end of the expandable member and the second diameter of the shaft in the recess is less than about 1.1 times the first diameter of the shaft.

16. The catheter of claim 14 wherein at least a portion of the proximal end of the expandable member is attached to the shaft in the recess such that at least a portion of the thickness of the proximal end of the expandable member and the second diameter of the shaft in the recess is less than about 1.05 times the first diameter of the shaft.

17. The catheter of claim 14 wherein at least a portion of the proximal end of the expandable member is attached to the shaft in the recess such that at least a portion of the thickness of the proximal end of the expandable member and the second diameter of the shaft in the recess no greater than about the first diameter of the shaft.

18. The catheter of claim 14 wherein at least a portion of the proximal end of the expandable member is attached to the shaft in the recess such that at least a portion of the thickness of the proximal end of the expandable member and the second diameter of the shaft in the recess is less than about 0.95 times the first diameter of the shaft.

19. The catheter of claim 14 wherein at least a portion of the proximal end of the expandable member is attached to the shaft in the recess such that at least a portion of the thickness of the proximal end of the expandable member and the second diameter of the shaft in the recess is less than about 0.9 times the first diameter of the shaft.

20. A balloon catheter comprising:
a head having at least two openings through which fluid may be passed;
a shaft extending from the head, the shaft having a first and second lumen disposed in communication with the at least two openings, the shaft further having an interior, an exterior, an outer diameter and a recess; and
a sleeve having a first end and a second end, each end of the sleeve having a thickness, wherein at least a portion of the first end of the sleeve is attached in a recess along the shaft such that the outer diameter of the shaft at the recess plus at least a portion of the thickness of the first end of the sleeve attached in the recess is less than about 1.15 times the outer diameter of the shaft immediately proximal the recess.

21. The catheter of claim 20 wherein an expandable cavity is formed between the sleeve and the shaft.

22. The catheter of claim 20 wherein at least a portion of the first end of the sleeve is attached to the exterior of the shaft, and at least a portion of the second end of the sleeve is attached to the interior of the shaft.

23. The catheter of claim 20 wherein at least a portion of the first end of the sleeve is attached to the exterior of the shaft, and at least a portion of the second end of the sleeve is attached to the exterior of the shaft.

24. The catheter of claim 20 wherein the catheter further comprises a tip attached to an end of the catheter shaft.

25. The catheter of claim 24 wherein at least a portion of the first end of the sleeve is attached to the exterior of the catheter shaft, and wherein at least a portion of the second end of the sleeve is attached to the tip.

26. A catheter comprising:
a head;
an elongate shaft having at least a first lumen extending longitudinally therethrough, a first end, a second end, an interior, an inside diameter and a recess; and
an expandable sleeve having a first end and a second end each end having an edge;
wherein at least a portion of one end of the sleeve is attached to the recess on the interior of the shaft such that the effective inside diameter of the shaft at the edge of the second end of the sleeve is at least about 90% of the inside diameter of the shaft immediately proximal the recess.

27. The balloon catheter of claim 26 wherein at least a portion of the first end of the sleeve is attached to the shaft so as to form a first cuff, and wherein the second end of the sleeve is attached to the shaft so as to form a second cuff.

28. The balloon catheter of claim 26 wherein at least a portion of the second end of the sleeve is attached to the second end of the shaft, and wherein the second end of the sleeve defines a portion of the passageway through the shaft.

29. The balloon catheter of claim 26 further comprising a tip attached to a second end of the shaft.

30. The balloon catheter of claim 29 wherein the sleeve is attached to the interior of the tip and the exterior of the shaft.

31. A catheter comprising:
a head;
an elongate shaft having at least a first lumen extending longitudinally therethrough, a first end, a second end, an interior, an inside diameter and a recess; and
an expandable sleeve having a first end and a second end each end having an edge;
wherein at least a portion of one end of the sleeve is attached to the recess on the interior of the shaft such that the effective inside diameter of the shaft at the edge of the end of the sleeve is no less than about 0.95 times the inside diameter of the shaft immediately proximal the recess.

32. The catheter of claim 31 wherein the effective inside diameter of the shaft at the edge of the end of the sleeve is no less than about 0.97 times the inside diameter of the shaft immediately proximal the recess.
